(19) United States
(12) Patent Application Publication
(10) Pub. No.: US 2009/0228011 A1
(43) Pub. Date: Sep. 10, 2009

(54) CRANIAL PERFORATOR

(76) Inventors: Victor B. Agbodoe, Stoughton, MA (US); Richard Densman, Lakeville, MA (US); Kenneth Hayes, Fall River, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/926,815

(22) Filed: Oct. 29, 2007

(51) Int. Cl. A61B 17/00

(52) U.S. Cl. 606/80

(57) ABSTRACT

A cranial perforator includes a generally cylindrical driver, a generally cylindrical drill member and a generally cylindrical sleeve therebetween. The driver is connected to the proximal end of the sleeve. The drill member is connected to the distal end of the sleeve. The sleeve has an internal clutch mechanism to selectively transfer rotational movement from the driver to the drill member. The drill member has an inner drill and an outer drill disposed about the inner drill. The inner drill has a distal end that has only two drilling flutes. A proximal end of the outer drill has a first predetermined diameter D1. A distal end of the outer drill has a second predetermined diameter D2, with the first predetermined diameter D1 being greater than the second predetermined diameter D2. A transition from the first predetermined diameter D1 to the second predetermined diameter D2 is a smooth, preferably radiused curve.

### BACKGROUND OF THE INVENTION

1. Field of the Invention

The present application generally relates to devices for drilling holes in the cranium.

2. Discussion of the Related Art

In many surgical operations it is necessary to obtain direct access to the cranial cavity and the brain. To perform such operations, it is often necessary to drill holes through the skull bone. Since the bone is very hard, it is necessary to apply significant pressure to drill through the bone, but it is also necessary to stop the drill once the bone has been penetrated to prevent damage to the dura.

A very satisfactory cranial drill with an automatic clutch mechanism for disengaging the drill when it approaches the interior layers of the skull bone or as it breaks through the interior of the skull is described in U.S. Pat. No. 4,456,010, issued Jun. 26, 1984, and U.S. Pat. No. 4,830,001, issued May 16, 1989, both of which are assigned to the assignee of the present invention, and the disclosures of which are fully incorporated by reference in their entirety in this application.

The invention described in U.S. Pat. No. 4,456,010 incorporates a sleeve about the exterior of the drill for holding the varius parts of the drill together during use. Sleeve 70 includes a raised portion 74 extending circumferentially on sleeve 70, partially thereabout and is axially aligned with a recess 14 on drill body 10. Raised portion 74 may be deformed radially inwardly to engage recess 14 on drill body 10 to hold sleeve 70 and driver 50 on drill body 10. Although that arrangement works satisfactorily, it was found that this prior drill could be improved by incorporating a retaining ring in slot 14 shown in the previous patent, assembling the sleeve 70 over drill body 10 and then affixing the retaining ring to the inside surface of the sleeve, preferably by means of ultrasonic welding, as taught by U.S. Pat. No. 4,830,001.

Both of these perforators have an inner drill and an outer drill, with the outer drill having an outside diameter that is about 14 mm. Surgeons have recently requested smaller size drills while still incorporating the internal clutch mechanism to selectively transfer rotational movement from the driver to the drill member so that the drill will automatically release when the drill penetrates the skull. In addition, surgeons have requested a drill profile that will permit the surgeon a clear view while drilling.

Accordingly, there remains a need for a perforator that can drill smaller size holes in the skull while still having a clutch mechanism to release the drill once the skull is penetrated and that also provide a clear view of the drilling site.

### SUMMARY OF THE INVENTION

In one embodiment, an exemplary cranial perforator includes a generally cylindrical driver, a generally cylindrical drill member and a generally cylindrical sleeve therebetween. The driver is connected to the proximal end of the sleeve. The drill member is connected to the distal end of the sleeve. The sleeve has an internal clutch mechanism to selectively transfer rotational movement from the driver to the drill member. The drill member has an inner drill and an outer drill disposed about the inner drill. The inner drill has a distal end that has only two drilling flutes. A proximal end of the outer drill has a first predetermined diameter D1. A distal end of the outer drill has a second predetermined diameter D2, with the first predetermined diameter D1 being greater than the second predetermined diameter D2. A transition from the first predetermined diameter D1 to the second predetermined diameter D2 is a smooth, preferably radiused curve.

### BRIEF DESCRIPTION OF THE DRAWINGS

Various exemplary embodiments disclosed herein will be more fully understood from the following detailed description taken in conjunction with the accompanying drawings, in which:

FIG. 1 is a perspective view of a cranial perforator in accordance with the present invention.

FIG. 2 is a side view of the cranial perforator with parts broken away;

FIG. 3 is a front view of the cranial perforator;

FIG. 4 is a side view of the distal end of the inner drill portion of the drill member;

FIG. 5 is a top view of the distal end of the inner drill portion of the drill member;

FIG. 6 is an enlarged front view of the inner drill portion of the drill member; and

FIG. 7 is a side view of the distal end of the inner drill portion of the drill member similar to FIG. 4, but taken from the opposite side.

### DETAILED DESCRIPTION OF THE INVENTION

Certain exemplary embodiments will now be described to provide an overall understanding of the principles of the structure, function, manufacture, and use of the devices disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope is defined solely by the claims. The features illustrated or described in connection with one exemplary embodiment may be combined with the features of other embodiments. Such modifications and variations are intended to be included within the scope of the present application.

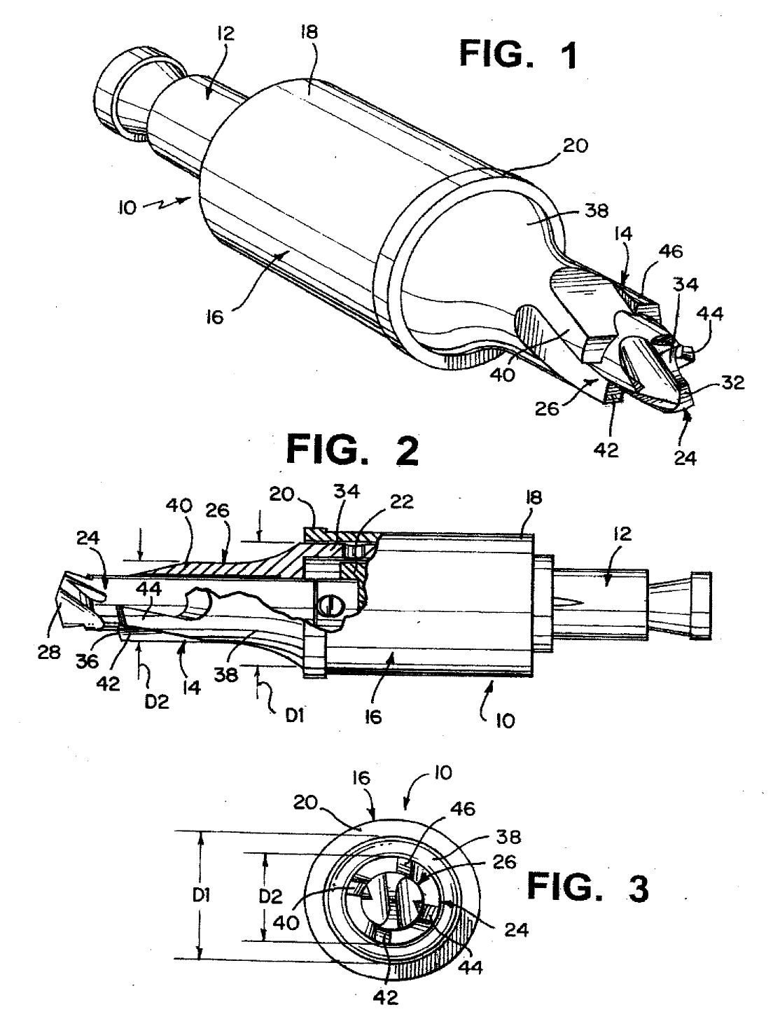

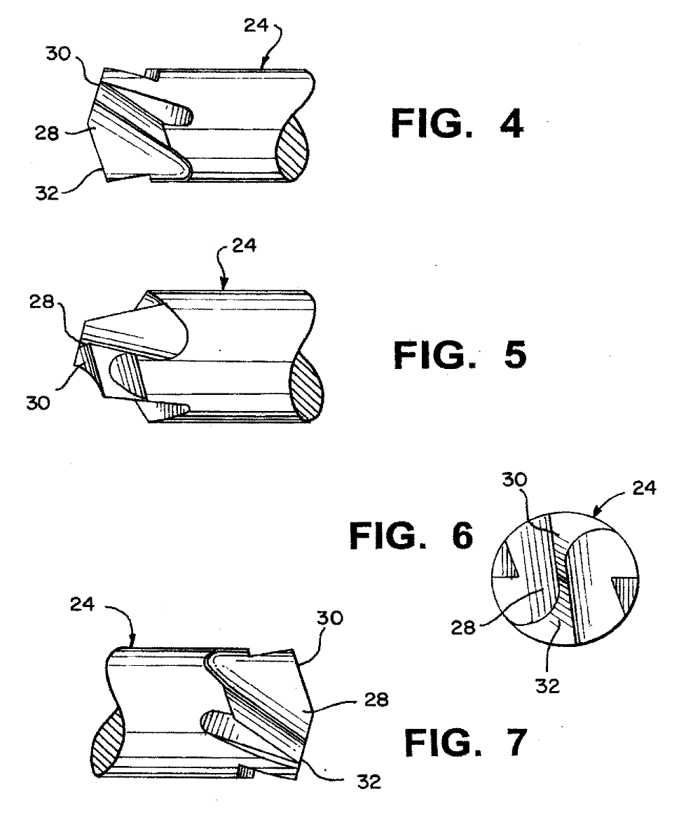

The present application generally provides devices for drilling holes in bone, especially the skull. An exemplary cranial perforator 10 is illustrated in FIGS. 1-7. Perforator 10 includes a generally cylindrical driver 12, a generally cylindrical drill member 14 and a generally cylindrical sleeve 16 therebetween. Sleeve 16 has a proximal end 18 and a distal end 20. Driver 12 is connected to the proximal end 18 of sleeve 16. Drill member 14 is connected to the distal end 20 of sleeve 16. Sleeve 16 has an internal clutch mechanism 22 to selectively transfer rotational movement from driver 12 to drill member 14 in a manner known to those skilled in the art. An example of such a clutch mechanism is disclosed in co-owned U.S. Pat. Nos. 4,456,010 and 4,830,001, the disclosures of which are hereby incorporated by reference in their entirety.

Drill member 14 has an inner drill 24 and an outer drill 26 disposed about inner drill 24. Inner drill 24 has a distal end 28 having only two drilling flutes 30, 32, which are preferably beveled and angled at about 15°. Thus, drilling flutes 30, 32 are adapted to cut human cranial bone. Outer drill 26 has a proximal end 34 and a distal end 36. Proximal end 34 of outer drill 26 has a first predetermined diameter D1, as shown in FIGS. 2 and 3. Distal end 36 of outer drill 26 has a second predetermined diameter D2, also as shown in FIGS. 2 and 3. Distal end 36 of outer drill 26 has four drilling flutes 40, 42, 44, 46. As shown, first predetermined diameter D1 is greater than second predetermined diameter D2. The transition 38 from first predetermined diameter D1 to second predetermined diameter D2 is a smooth curve, and preferably a radiused curve. Transition 38 is disposed proximate to the proximal end 34 of outer drill 26. In a currently preferred exemplary embodiment, the first predetermined diameter is about 9 mm so that a relatively small hole can be created in the skull. In a currently preferred exemplary embodiment, a ratio of the second predetermined diameter D2 to the first predetermined diameter D1 is about 0.64. Thus, perforator 10 in accordance with the present invention is capable of drilling smaller size holes in the skull while still having a clutch mechanism to release the drill once the skull is penetrated. The use of only two flutes on the inner drill in combination with four flutes on the outer drill allows for cranial bone removal without binding and provides a clean cut when drilling smaller size holes. In addition, the smooth curve transition to smaller diameter D2 near the proximal end of the outer drill provides the surgeon with a clear view of the drilling site.

A person skilled in the art will appreciate that the various devices disclosed herein can be formed from a variety of materials. Moreover, particular components can be implantable and in such embodiments the components can be formed from various biocompatible materials known in the art. Exemplary biocompatible materials include, by way of non-limiting example, composite plastic materials, biocompatible metals and alloys such as stainless steel, titanium, titanium alloys and cobalt-chromium alloys, glass, and any other material that is biologically compatible and non-toxic to the human body.

One skilled in the art will appreciate further features and advantages based on the above-described embodiments. Accordingly, the disclosure is not to be limited by what has been particularly shown and described, except as indicated by the appended claims. All publications and references cited herein are expressly incorporated herein by reference in their entirety.

What is claimed is:

1. A cranial perforator comprising:
a generally cylindrical driver;
a generally cylindrical drill member;
a sleeve having a proximal end and a distal end; said driver being connected to said sleeve proximal end, said drill member being connected to said sleeve distal end, said sleeve having an internal clutch to selectively transfer rotational movement from said driver to said drill member;
wherein said drill member having an inner drill and an outer drill disposed about said inner drill, said inner drill having a distal end having only two drilling flutes adapted to cut human cranial bone, said outer drill having a proximal end and a distal end, said proximal end of said outer drill having a first predetermined diameter, said distal end of said outer drill having a second predetermined diameter, said first predetermined diameter being greater than said second predetermined diameter, a transition from said first predetermined diameter to said second predetermined diameter being a smooth curve.

2. The perforator of claim 1, wherein the transition is a radiused curve.

3. The perforator of claim 1, wherein the transition is disposed proximate to said proximal end of said outer drill.

4. The perforator of claim 2, wherein the transition is disposed proximate to said proximal end of said outer drill.

5. The perforator of claim 1, wherein a ratio of said second predetermined diameter to said first predetermined diameter is about 0.64.

6. The perforator of claim 1, wherein the first predetermined diameter is about 9 mm.

7. The perforator of claim 5, wherein the first predetermined diameter is about 9 mm.

8. The perforator of claim 1, wherein the first predetermined diameter is less than or equal to about 9 mm.

9. The perforator of claim 5, wherein the first predetermined diameter is less than or equal to about 9 mm.

10. The perforator of claim 1, wherein the outer drill has four drilling flutes.

11. The perforator of claim 6, wherein the outer drill has four drilling flutes.
